(19) United States
(12) Patent Application Publication
(10) Pub. No.: US 2004/0067266 A1
(43) Pub. Date: Apr. 8, 2004

(54) Weight loss compound

(76) Inventor: Frank R. Toppo, Las Vegas, NV (US)

Correspondence Address: FRANK R. TOPPO, MD, 2101 PALM CANYON COURT, LAS VEGAS, NV, 89117, US

(21) Appl. No.: 10/265,372

(22) Filed: Oct. 7, 2002

(51) Int. Cl. A61K 35/78

(52) U.S. Cl. 424/730; 424/747; 424/765; 424/777

(57) ABSTRACT

The delivery of St. John's Wort coupled separately with Natural Peppermint, Banana and Green Apple flavors via the nasal and or buccal mucosa in a ph appropriate solution, in homeopathic doses, is effective as an anorexant.

### SPECIFICATION

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

Obesity has become a national epidemic. According to a recent report by the United States Surgeon General, 61% of American adults are either overweight or obese. For the first time in our nations history, childhood obesity has become a major health concerns.

To date, nothing has been shown to be effective in long-term management of obesity.

Two pharmacologic options are available today for safely treating depression-the many antidepressants approved by the United States Food and Drug Administration (FDA) and St. John's Wort, an older herbal remedy made from the yellow flowers of the widespread perennial herb Hypericum perforatum. This herbal remedy has been termed natural Prozac.

H. perforatum was named St. John's Wort by early Christians because this plant begins blooming on June 24^{th}, the feast of St. John the Baptist. Wort is the Old English term for plant. In the mid-19^{th} century, snake oil salesmen sold the leaves and flowers of this plant as a cure for “low spirits.”

St. John's Wort's primary indication is mild to moderate depression. More than 6,000 patients in more than 425 studies have been conducted to ascertain the effectiveness of this herb in treating depression, making it the most studied herbal preparation, ever!

Prozac type medications and St. Johns Wort have been successful in treating depression and obesity, which affects tens of millions of Americans, young and old alike.

The research has clearly shown that St. John's Wort performs at least as well as the tricyclic antidepressants (with many fewer side effects) and possibly as well as fluoxetive, sertraline and other drugs in this class. The daily dosage used in these studies was 900 mg to 1800! At these large dosages, people being treated with AIDS drugs and those who are taking medication (cyclosporine) used to prevent kidney rejection, are at some risk.

Also, it has been shown that certain scents have exerted a positive anorexic effect on many obese patients. Specifically, Green Apple, Peppermint and Banana have been shown to be effective.

### BRIEF SUMMARY OF THE INVENTION

Currently, the St. John's Wort extract is delivered in pill form. This means the active ingredients must be extracted from the pill within the gastrointestinal tract. The action of the acidic juices of the stomach and then the alkaline juices of the small intestine decrease the amount of St. John's Wort available for absorption. This also delays the effect of the St. John's Wort. By delivering the St. John's Wort directly to the nasal/buccal mucosa, absorption and effect are immediate. Also, much lower doses of St. John's Wort may be used. And the delivery of the scents (Green Apple, Banana and Peppermint) have until now been all but impossible except under very strict, controlled circumstances. The delivery of these scents via the nasal or buccal mucosa is convenient, effective and immediate.

Delivery System

These delivery systems allow for a much lower amount of St. John's Wort in order to be effective. And because these delivery systems need not go through the gastrointestinal system, not only is the dose of St. John's Wort and flavors much less, the effect of these combinations is immediate.

### DETAILED DESCRIPTION OF THE INVENTION

Because of the large doses necessary for St. John's Wort to be effective when taken orally, we have developed three delivery systems, which greatly decrease the amount necessary for the combination of St. John's Wort and the three flavors to be effective.

St. John's Wort by itself or in combination with either Peppermint, Green Apple or Banana are delivered into the nasal mucosa by dropper, nasal inhalant or metered dose injector, etc, and a sublingual spray which delivers a metered dose to the buccal mucosa. And finally the individual oral slips similar to the current breath strips. Each strip will contain the St. John's Wort in a homeopathic dosage of 10× to 1×. 1× obviously being more effective. The nasal and oral sprays are ph factored and in a solution of, but not limited to, USP Purified Water, USP Vegetable glycerin and Natural Peppermint, Banana and Green Apple flavors.

The oral strips are made from combinations of, but not limited to, Tapioca, gelatin, glycerin, cellulose, lecithin, Xantham gum, Natural Peppermint, Banana and Green Apple flavors, etc.

Each system must be ph compatible to the mucosa of the nose and sublingual areas in order for the active ingredients (St. John's Wort and Natural flavors) to be absorbed and not to be an irritant to the tissue.

### DETAILED DESCRIPTION OF THE INVENTION

Because of the large doses necessary for St. John's Wort to be effective when taken orally, we have developed three delivery systems, which greatly decrease the amount necessary for the combination of St. John's Wort and the three flavors to be effective.

St. John's Wort by itself or in combination with either Peppermint, Green Apple or Banana are delivered into the nasal mucosa by dropper, nasal inhalant or metered dose injector, etc, and a sublingual spray which delivers a metered dose to the buccal mucosa. And finally the individual oral slips similar to the current breath strips. Each strip will contain the St. John's Wort in a homeopathic dosage of 10× to 1×. 1× obviously being more effective. The nasal and oral sprays are ph factored and in a solution of, but not limited to, USP Purified Water, USP Vegetable glycerin and Natural Peppermint, Banana and Green Apple flavors.

The oral strips are made from combinations of, but not limited to, tapioca, gelatin, glycerin, cellulose, lecithin, xantham gum, natural peppermint, banana and green apple flavors, etc.

Each system must be ph compatible to the mucosa of the nose and sublingual areas in order for the active ingredients (St. John's Wort and Natural flavors) to be absorbed and not to be an irritant to the tissue.

1. What I claim as my invention is:
The combination of St. John's Wort and Natural Peppermint, Banana and Green Apple delivered via the nasal or buccal mucosa.
